(19) United States
(12) Patent Application Publication
(10) Pub. No.: US 2016/0287060 A1
(43) Pub. Date: Oct. 6, 2016

(54) IMAGING MODULE AND ENDOSCOPE

(71) Applicant: FUJIKURA LTD., Tokyo (JP)

(72) Inventors: Hideaki USUDA, Sakura-shi (JP); Kazuhiro DOMOTO, Sakura-shi (JP); Kenichi ISHIBASHI, Sakura-shi (JP)

(73) Assignee: FUJIKURA LTD., Tokyo (JP)

(21) Appl. No.: 15/070,305

(22) Filed: Mar. 15, 2016

(30) Foreign Application Priority Data
Mar. 31, 2015 (JP) 2015-073174

(51) Int. Cl. A61B 1/05; A61B 1/00; A61B 1/005; H04N 5/225

(52) U.S. Cl. CPC A61B 1/051; H04N 5/2252; A61B 1/00114; A61B 1/005; A61B 1/00163; A61B 1/00071

(57) ABSTRACT

An imaging module of the invention including a flexible wiring substrate that includes: a first end portion including a connector; a second end portion including a mount portion; a mounting portion on which an imaging device is mounted; a first bend portion bended and provided between the mounting portion and the first end portion; and a second bend portion bended and provided between the mounting portion and the second end portion. The first end portion and the second end portion overlap each other at a rear side opposite to an imaging surface of the imaging device, a cable is connected to a surface of the first end portion on the opposite side of the second end portion, and a capacitor is mounted on a surface of the second end portion on the opposite side of the first end portion.

### CROSS REFERENCE TO RELATED APPLICATIONS

This application claims priority from Japanese Patent Application No. 2015-073174 filed on Mar. 31, 2015, the contents of which are incorporated herein by reference.

### BACKGROUND OF THE INVENTION

1. Field of the Invention

The present invention relates to an imaging module and an endoscope.

2. Description of the Related Art

Japanese Unexamined Patent Application, First Publication No. 2011-217887 (hereinbelow, Patent Document 1) discloses a structure including a flexible substrate provided with an imaging device in which a through-hole interconnection is formed. In the structure, both sides of the flexible substrate are bended toward the opposite side of the imaging surface so as not to exceed the range of the outer shape of the device, a component is mounted on the substrate, the mount portion is further bended to the inside of the structure, and the component also does not exceed the range of the outer shape.

Japanese Unexamined Patent Application, First Publication No. 2013-214815 (hereinbelow, Patent Document 2) discloses a structure including a flexible substrate provided with an imaging device thereon. In the structure, both sides of the flexible substrate are bended toward the opposite side of the imaging surface so as to protrude from the range of the outer shape of the device, both end portions of the flexible substrate come close to each other, and the bending shape of the flexible substrate is fixed by filling the region surrounded by the flexible substrate with an adhesive.

In Patent Document 1, in order to fix the substrate end of the flexible substrate to a cable, a component is mounted on the surface on which the substrate is fixed to a block. Accordingly, this structure is not suited to reduce an imaging module in size (in particular, reduction in diameter around an optical axis). Particularly, it is difficult to reduce the diameter to be less than or equal to 2 mm.

The structure disclosed in Patent Document 2 is suited to reduce the diameter; however, it is difficult to ensure a space in which a component is mounted on the flexible substrate.

### SUMMARY OF THE INVENTION

Some aspects of the invention were conceived in view of the above-described circumstances and have an object thereof to provide an imaging module that provides a capacitor at a distal end of an endoscope and can reduce the diameter thereof, and an endoscope provided with the imaging module.

In order to solve the above-described problem, an imaging module according to a first aspect of the invention includes: an imaging device; a flexible wiring substrate on which the imaging device is mounted; a cable connected to the flexible wiring substrate; and a capacitor mounted on the flexible wiring substrate, wherein the flexible wiring substrate includes: a first end portion including a connector which is connected to the cable; a second end portion including a mount portion on which the capacitor is mounted; a mounting portion on which the imaging device is mounted between the first end portion and the second end portion; a first bend portion bended and provided between the mounting portion and the first end portion; and a second bend portion bended and provided between the mounting portion and the second end portion, the first end portion and the second end portion overlap each other at the rear side opposite to an imaging surface of the imaging device, the cable is connected to a surface of the first end portion on the opposite side of the second end portion, and the capacitor is mounted on a surface of the second end portion on the opposite side of the first end portion.

In the imaging module according to the first aspect of the invention, the first end portion and the second end portion may be adhesively attached to each other and may be formed in a planar shape.

The imaging module according to the first aspect of the invention may include a lens provided on the imaging surface of the imaging device.

The imaging module according to the first aspect of the invention may include an insulating tube that surrounds the first end portion and the second end portion.

In the imaging module according to the first aspect of the invention, the periphery of the imaging device may be covered with a light shielding material.

In the imaging module according to the first aspect of the invention, the height of the capacitor from the second end portion may be less than or equal to a half of the internal diameter of a virtual cylindrical body which is capable of entirely accommodating the imaging device and the flexible wiring substrate.

An endoscope according to a second aspect of the invention uses the imaging module according to the first aspect of the invention.

The endoscope according to the second aspect of the invention may include a sleeve that accommodates the imaging module therein.

In the endoscope according to the second aspect of the invention, the height of the capacitor from the second end portion may be less than or equal to a half of the internal diameter of the sleeve.

### EFFECTS OF THE INVENTION

According to the invention, it is possible to provide an imaging module that provides a capacitor at a distal end of an endoscope and can reduce the diameter thereof, and an endoscope provided with the imaging module.

### BRIEF DESCRIPTION OF THE DRAWINGS

FIG. 1 is a side view showing an example of an imaging module of the invention.

FIG. 2 is a side view showing an example of an imaging module provided with a lens and an insulating tube.

FIG. 3 is a cross-sectional side view showing an example of a distal end of an endoscope.

FIG. 4 is a cross-sectional plan view showing an example of a distal end of an endoscope.

### DETAILED DESCRIPTION OF THE INVENTION

Hereinafter, preferred embodiments of the invention will be described with reference to drawings.

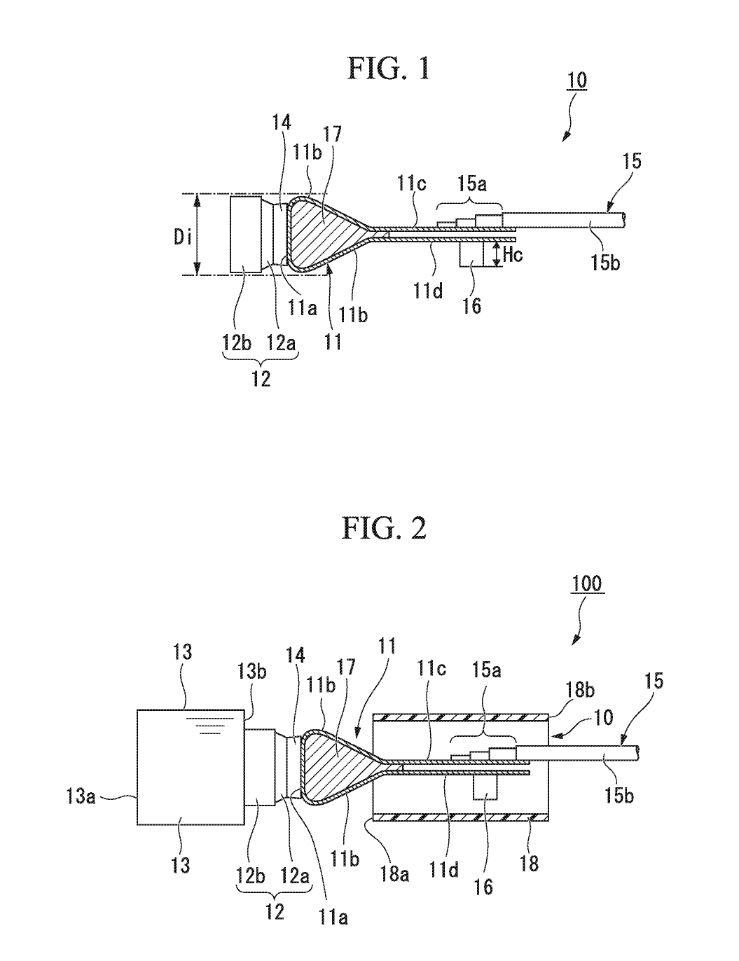

FIG. 1 shows an example of imaging module 10 according to the embodiment. The imaging module 10 includes a flexible wiring substrate 11, an imaging device 12, cables 15, and a capacitor 16. The flexible wiring substrate 11 includes: a mounting portion 11a on which the imaging device 12 is mounted; two bend portions 11b which are bended at both sides of the mounting portion 11a; a first end portion 11c including a connector which is connected to the cables 15; and a second end portion 11d including a mount portion on which the capacitor 16 is mounted.

The imaging device 12 includes an imaging device main body 12a and a protector 12b provided on the imaging surface thereof. As the protector 12b, a cover glass, a coating, a layer formed by applying a liquid form material, or the like is adopted. As long as the imaging device (main body) is a device that detects light and generates an electrical signal producing an image, the kind of imaging device is not particularly limited. As the above-described device, a solid-state image sensing device (CMOS, CCD, or the like) including a semiconductor device which is provided in a light receiving portion is preferably used because the size thereof is small. As other examples, an organic imaging device including an organic optical material which is provided in the light receiving portion, an image pick-up tube including an electron tube which is provided in the light receiving portion, or the like may be adopted.

In a state where the flexible wiring substrate 11 is developed in a plan view, the flexible wiring substrate 11 is formed in a band shape such that the flexible wiring substrate 11 has the first end portion 11c and the second end portion 11d which are located at both end portions thereof in the longitudinal direction thereof, and the mounting portion 11a is provided between the first end portion 11c and the second end portion 11d. A first bend portion 11b is provided between the mounting portion 11a and the first end portion 11c, and a second bend portion 11b is provided between the mounting portion 11a and the second end portion 11d.

As shown in FIG. 1, in a state where the imaging module 10 is formed by assembling parts thereof, the first end portion 11c and the second end portion 11d overlap each other at the rear side (right side of FIG. 1) which is opposite to the imaging surface of the imaging device 12. The first end portion 11c has both surfaces (a first surface and a second surface) on the flexible wiring substrate 11, the cables 15 are connected to the first surface of the first end portion (upper surface shown in FIG. 1) which is on the opposite side of the second surface facing the second end portion 11d. Additionally, the second end portion 11d has both surfaces (a first surface and a second surface) on the flexible wiring substrate 11, the capacitor 16 is connected to the first surface (lower surface shown in FIG. 1) which is on the opposite side of the second surface facing the first end portion 11c.

In a state where the imaging module 10 is formed by assembling the parts thereof, it is preferable that a fixed portion 17 be provided in a substantially triangular-shaped space surrounded by the mounting portion 11a and the two bend portions 11b by use of an adhesive, a resin, or the like. As a result of providing the fixed portion 17, the shape of the mounting portion 11a and the two bend portions 11b is stabilized, two end portions 11c and 11d are held by the fixed portion, and a degree of operatability is improved in the case of carrying out an operation of forming the imaging module 10 by assembling and attaching the parts thereto. The fixed portion 17 may be formed so as to fill the space surrounded by the flexible wiring substrate 1 and may be formed in part of the space. It is preferable that the two bend portions 11b be inclined so as to come close to each other in the direction toward the two end portions 11c and 11d from the mounting portion 11a in many cases.

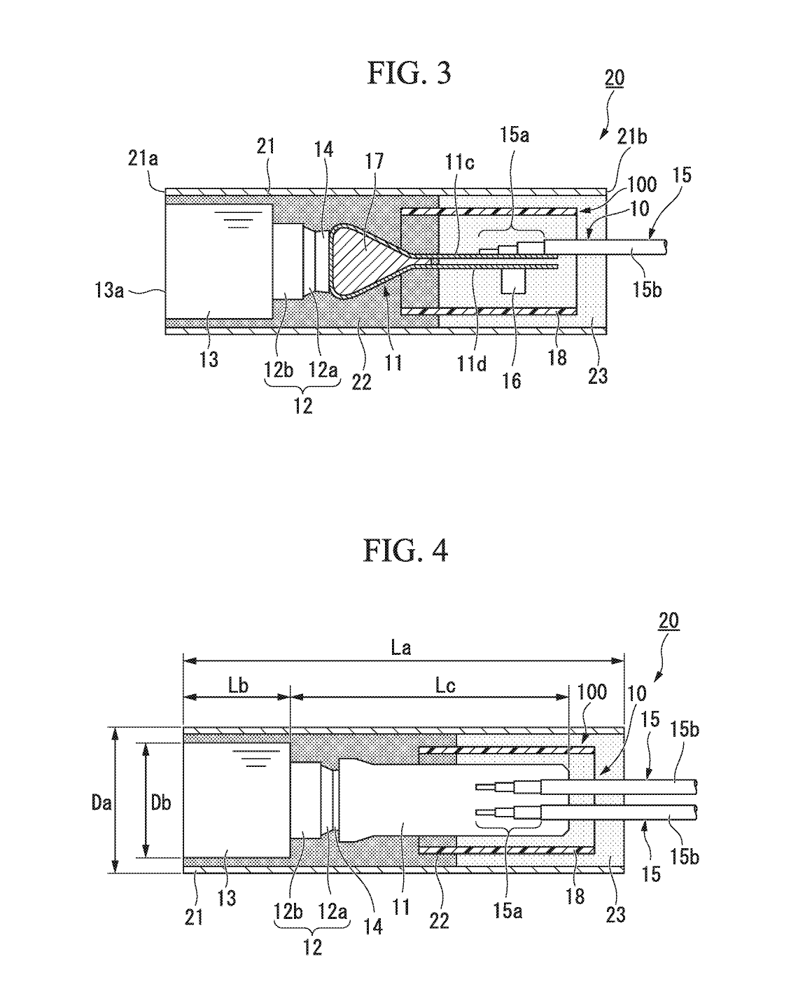

Since the two end portions 11c and 11d come close to each other, it is possible to ensure a space in which the cables 15 are connected to the first end portion 11c and the capacitor 16 is mounted on the second end portion 11d in the height direction of the mounting portion 11a (vertical direction in FIG. 1). Accordingly, as shown in FIG. 3, in the case where the imaging module 10 is provided on a distal end 20 of an endoscope (which will be particularly described later), it is possible to realize a reduction in the size of the imaging module 10 and reduction of the diameter thereof.

The first end portion 11c and the second end portion 11d may be adhesively attached to each other. Furthermore, a reinforcing agent (adhesive, coating, or the like) that reinforces the flexible wiring substrate 11 may be provided: between the two end portions 11c and 11d; or on one or both surfaces of the two end portions 11c and 11d, so as to maintain the two end portions 11c and 11d in a planar shape.

The flexible wiring substrate 11 is a printed-wiring substrate using a flexible insulating substrate. In other cases, a flex-rigid wiring board having an insulating substrate partially having a hard portion, a printed circuit board having printed components, for example, FPC or the like can be adopted. A material used to form the insulating substrate is not particularly limited. As the material, an insulating resin such as polyimide, polyester, or liquid crystal polymer is adopted. As a conductor provided on a wiring board, wiring, grounded conductor, dummy conductor, or the like is adopted. As a material used to form the above-mentioned conductors, a metal such as Cu. Ni, Au, Ag, Sn, Ti, or Cr, an alloy, a composite, or a layered body which include one or more of the above-described metals is adopted.

Wirings are provided on one surface or on both surfaces of the flexible wiring substrate 11. The wirings electrically connect electrical components such as the imaging device 12, the cables 15, or the capacitor 16. For example, as shown in FIG. 1, in the case where the flexible wiring substrate is bended, the wirings may be provided on the external surface thereof so as to form a pattern. The structure that electrically connects the wirings of the flexible wiring substrate 11 and electrodes of the imaging device 12 on the mounting portion 11a is not particularly limited. A conducting bump such as a solder, wire bonding, through holes that allow terminals of the imaging device 12 to pass therethrough, or the like may be provided on the flexible wiring substrate 11.

The imaging device 12 can be fixed to the mounting portion 11a of the flexible wiring substrate 11 or reinforced by a fixing agent 14 such as an adhesive. A conductor is exposed at the front-end portion 15a of the cable 15. Soldering, application of an electroconductive adhesive, or the like is adopted as a method of connecting the wirings of the flexible wiring substrate 11 to the cables 15, the capacitor 16, or the like.

Each cable 15 is a lengthy body having a signal transmission function. As a specific example, an electrical cable such as a coaxial cable is adopted as the cable 15. In the drawing shown as an example, two cables 15 are provided in parallel with each other. The electrical signal transmitted by the cable 15 may be a balanced signal or an unbalanced signal. In the case of using a coaxial cable as the cable, one cable includes an internal conductor and an external conductor, the internal conductor and the external conductor are connected to different terminals provided on the flexible wiring substrate 11 by soldering or the like.

The capacitor 16 is a passive element having electrostatic capacitance (capacitance) and is referred to as a condenser. For example, in the case of providing a capacitor for decoupling in order to avoid variation in voltage, it is possible to reduce noise. Additionally, as a power storage which is used when stroboscopic light emission is carried out, a capacitor can also be provided. In the other cases, as the purpose of using a capacitor, a capacitor may be used for: coupling; filtering; or smoothing a source. Since the imaging module 10 according to the embodiment has the space (height) which is used to mount the capacitor 16 on the second end portion 11d, even in an endoscope having a reduced diameter, it is possible to sufficiently ensure electrostatic capacitance.

In terms of reduction in the size of the imaging module 10, the height Hc of the capacitor 16 from the second end portion 11d is preferably less than or equal to half of the internal diameter Di of a virtual cylindrical body that can entirely accommodate the imaging device 12 and the flexible wiring substrate 11 therein. Here, the internal diameter Di of the virtual cylindrical body means that the internal diameter of an arbitrary cylindrical body (particularly, the minimum diameter thereof) which can accommodate the imaging device 12 and the flexible wiring substrate 11 therein and which excludes the capacitor 16 therefrom. For example, as shown in FIG. 1, the internal diameter Di can be the internal diameter required to accommodate the imaging device 12, the mounting portion 11a on which the imaging device is mounted, and the bend portions 11b therein. The internal diameter Di may be different from the internal diameter of a sleeve (for example, reference numeral 21 shown in FIG. 4) which is used to practically accommodate the imaging module 10 therein.

FIG. 2 shows an example of an imaging module 100 provided with a lens unit 13 and an insulating tube 18. In this example, the lens unit 13 which has a lens built therein and is provided on the imaging surface of the imaging device 12 (particularly, provided on the protector 12b) is provided in the imaging module. The external appearance of the lens unit 13 is formed in a cylindrical shape having a front-end face 13a and a rear-edge face 13b which serve as both bottom faces, and the lens unit 13 is fixed so that the rear-edge face 13b faces the imaging device 12. In terms of reduction in the size of the imaging module 10, the height Hc (refer to FIG. 1) of the capacitor 16 from the second end portion 11d is preferably less than or equal to a half of the outer diameter of the lens unit 13. Here, in the case where the shape of the lens unit 13 is not cylindrical, the diameter of the circumscribing circle can be regarded as the outer diameter.

The inner structure of the lens unit 13 is not particularly limited. In the inner structure, an objective optical system is formed which includes one, two or more lenses in a tubular holder (external cylinder, lens barrel). The lens may include the combination of the same kind of or the different kind of lenses selected from a convex lens, a concave lens, a concave-convex lens, or the like. As the outer shape of the lens unit 13, cylindrical shape, square tubular shape, or the like is adopted. Moreover, the lens may directly come into contact with the protector 12b of the imaging device 12, and an air space or a resin layer may be provided between the lens and the protector 12b.

The insulating tube 18 is arranged so as to surround the first end portion 11c and the second end portion 11d of the flexible wiring substrate 11. The insulating tube 18 is disposed in the longitudinal direction of the cables 15. The bend portions 11b and the mounting portion 11a of the flexible wiring substrate 11 may be disposed outside the insulating tube 18 so as to protrude from the front-end 18a of the insulating tube 18. Part of or the entire of the bend portions 11b and the mounting portion 11a may be disposed inside the insulating tube 18. The front-end portion 15a at which the conductor of the cable 15 is exposed is accommodated in the insulating tube 18, and a coated portion 15b having an insulating coating protrudes from a back end 18b of the insulating tube 18. Part of the coated portion 15b may be disposed inside the insulating tube 18. A material used to form the insulating tube 18 is not particularly limited. As the material, various resins such as silicone, soft polyvinyl chloride, elastomer, polyolefin, fluorine resin; a rubber; or the like are adopted. The insulating tube 18 may be fixed to the flexible wiring substrate 11 by applying an adhesive or the like therebetween.

A method of manufacturing the imaging modules 10 and 100 is not particularly limited. For example, firstly, a planar flexible wiring substrate 11 is prepared, it is only necessary to attach an imaging device 12, a lens unit 13, cables 15, a capacitor 16, or the like to the flexible wiring substrate in suitable order. The bend portions 11b may be formed before or after the imaging device 12 is connected to the mounting portion 11a. In the case of connecting the imaging device 12 to the mounting portion 11a before the bend portions 11b are formed, since the flexible wiring substrate 11 is formed in a planar shape, a connection operation can be easily carried out. After the two bend portions 11b are formed, before attachment of the other components (the lens unit 13, the cables 15, and the capacitor 16), it is preferable to form the fixed portion 17 by filling the space surrounded by the mounting portion 11a and the two bend portions 11b with the material thereof. As a material filling the space as the fixed portion 17, a curable resin such as a thermosetting resin or a light curable resin is preferably used.

The aforementioned imaging modules 10 and 100 can be used in an endoscope. FIGS. 3 and 4 show that an example of the imaging module 100 including the lens unit 13 and the insulating tube 18 is accommodated in a sleeve 21 located at the distal end 20 of an endoscope. The intended use of the endoscope is not limited to a medical use in which the endoscope is inserted into a living body. The endoscope is applicable to observe the inside of a device, a building, a tube, various structural objects, or the like.

The sleeve 21 can be made of a metal, a plastic, a ceramics, a composite material, or the like. For example, a metal sleeve such as a stainless steel is adopted. As a result of accommodating the entirety of or part of the imaging modules 10 and 100 into the hard sleeve 21, it is possible to maintain a function of the imaging module for a longer time. The sleeve 21 shown as an example in the drawing opens in two directions, that is, only at a front end 21a and a back end 21b. The sleeve 21 is formed in a cylindrical shape such that the side surface thereof is uniformly closed. The member that accommodates the imaging modules 10 and 100 therein is not only a cylindrical sleeve but also a frame-shaped member, a grid-shaped member, or the like. In the case where a material used to form the sleeve 21 is opaque with respect to the wavelength detected by the imaging device 12, it is possible to prevent external light from entering the sleeve 21, and it is therefore preferable.

In the case where the sleeve 21 is made of a conductor such as a metal, it is preferable to electrically insulate the flexible wiring substrate 11 from the conductors of the cables 15 of the imaging modules 10 and 100. In the embodiment, the conductors of the imaging modules 10 and 100 are insulated from the sleeve 21 with the insulating tube 18, filler 23, or the like interposed therebetween. Regarding the insulator used in this embodiment, the insulator is not limited to this. As other insulators, a structure of using any one of, or two or more of the insulating tube 18, a light shielding material 22, or the filler 23, a structure in which the inner surface of or the entirety of the sleeve 21 is made of an insulating material, a structure in which an insulating coating is provided on the sleeve 21 or the surface of the flexible wiring substrate 11, or the like is adopted.

When the capacitor 16 is accommodated in the sleeve 21, in order to prevent an excessive force from being applied between the cable 15 and the flexible wiring substrate 11, it is preferable that the two end portions 11c and 11d be located adjacent to the center in the radial direction of the sleeve 21 (direction perpendicular to the axis thereof). Consequently, it is preferable that the height Hc of the capacitor 16 from the second end portion 11d (refer to FIG. 1) be less than or equal to a half of the internal diameter of the sleeve 21.

It is preferable that the periphery of the imaging device 12 be covered with a light shielding material. In the case where a light shielding material is not provided on the external surface of the imaging device 12, particularly, in the case where the protector 12b is transparent, as a result of providing the light shielding material 22 on the periphery of the imaging device 12, it is possible to prevent external light from entering the imaging device. In the case of the distal end 20 of the endoscope according to the embodiment, the portion (region) near the front end 21a of the sleeve 21 is filled with the light shielding material 22. As the light shielding material 22, a composition is adopted which contains a medium such as a resin or an adhesive into which a light absorptive material, for example, carbon, black pigment, or the like is mixed. In the case where the external cylinder of the lens unit 13 has a light shielding property, it is not necessarily the case that the light shielding material 22 is provided around the lens unit 13. The light shielding material 22 may be used to double as a filler that stabilizes the posture (position and direction) of the lens unit 13 inside the sleeve 21.

In order to stabilize the postures (position and direction) of the flexible wiring substrate 11 and the front-end portion 15a of the cable 15, the filler 23 is provided at the portion (region) near the back end 21b of the sleeve 21. In the case where there is a possibility that the filler 23 comes into contact with a conductor, the filler preferably has an electrical insulation property. A method of forming the light shielding material 22 and the filler 23 is not particularly limited. After the imaging modules 10 and 100 are inserted into the sleeve 21, a material in a flow condition may be injected into the sleeve 21 and the material may be cured. Before the imaging modules 10 and 100 is inserted into the sleeve 21, part of or the entirety of the material may be applied to the portion (region) near the sleeve 21 or the portion (region) near the imaging modules 10 and 100.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

In the imaging module according to the embodiment, since the diameter thereof is easily reduced, and it is thereby preferable to use the imaging module in an endoscope; however, the invention is not limited to this embodiment, and the imaging module is applicable to an optical observation device used in various devices, vehicles, robots, or the like, an optical detection device, or the like.

### Example

FIGS. 3 and 4 show specific example of the imaging module 100 and the distal end 20 of the endoscope, particularly, show the sizes (length, diameter) thereof. The diameter Da of the sleeve 21 is in the range of 1.4 to 2.0 mm, the diameter Db of the lens unit 13 is in the range of 1.0 to 1.5 mm, the length La of the sleeve 21 is in the range of 3.5 to 7.0 mm, the length Lb of the lens unit 13 is in the range of 1.0 to 1.5 mm, and the overall length 1.c (length from the front end of the imaging device 12 to the back end of the flexible wiring substrate 11) of the imaging module 10 is in the range of 3.0 to 4.0 mm. In other cases, the invention is not limited to the specific example above.

What is claimed is:

1. An imaging module comprising:
an imaging device;
a flexible wiring substrate on which the imaging device is mounted;
a cable connected to the flexible wiring substrate; and
a capacitor mounted on the flexible wiring substrate, wherein
the flexible wiring substrate comprises:
a first end portion including a connector which is connected to the cable;
a second end portion including a mount portion on which the capacitor is mounted;
a mounting portion on which the imaging device is mounted between the first end portion and the second end portion;
a first bend portion bended and provided between the mounting portion and the first end portion; and
a second bend portion bended and provided between the mounting portion and the second end portion,
the first end portion and the second end portion overlap each other at a rear side opposite to an imaging surface of the imaging device,
the cable is connected to a surface of the first end portion on the opposite side of the second end portion, and
the capacitor is mounted on a surface of the second end portion on the opposite side of the first end portion.

2. The imaging module according to claim 1, wherein
the first end portion and the second end portion are adhesively attached to each other and are formed in a planar shape.

3. The imaging module according to claim 1, further comprising:
a lens provided on the imaging surface of the imaging device.

4. The imaging module according to claim 1, further comprising:
an insulating tube that surrounds the first end portion and the second end portion.

5. The imaging module according to claim 1, wherein
a periphery of the imaging device is covered with a light shielding material.

6. The imaging module according to claim 1, wherein
a height of the capacitor from the second end portion is less than or equal to a half of an internal diameter of a virtual cylindrical body which is capable of entirely accommodating the imaging device and the flexible wiring substrate.

7. An endoscope comprising:
the imaging module according to claim 1.

8. The endoscope according to claim 7, further comprising:
a sleeve that accommodates the imaging module therein.

9. The endoscope according to claim 8, wherein
a height of the capacitor from the second end portion is less than or equal to a half of an internal diameter of the sleeve.
